(19) United States
(12) Patent Application Publication
(10) Pub. No.: US 2024/0304007 A1
(43) Pub. Date: Sep. 12, 2024

(54) METHOD AND SYSTEM FOR THE SEGMENTATION AND CLUSTERING OF NUCLEI BASED ON SINGLE-CELL PATHOLOGICAL IMAGES

(71) Applicant: WUXI NO. 2 PEOPLE'S HOSPITAL, WUXI (CN)

(72) Inventors: NINGHAN FENG, WUXI (CN); HONG TANG, WUXI (CN); GUANZHEN YU, WUXI (CN); YUEZHOU ZHANG, WUXI (CN); YANGKUN FENG, WUXI (CN); FENGPING LIU, WUXI (CN); YANG WANG, WUXI (CN); PENG JIANG, WUXI (CN)

(21) Appl. No.: 18/614,717

(22) Filed: Mar. 24, 2024

(30) Foreign Application Priority Data
Mar. 24, 2023 (CN) 202310293754.X

(51) Int. Cl. G06V 20/69; G06T 7/00; G06T 7/11; G06V 10/44; G06V 10/46; G06V 10/762; G16H 30/40; H04N 1/40

(52) U.S. Cl. CPC G06V 20/695; G06T 7/0012; G06T 7/11; G06V 10/44; G06V 10/46; G06V 10/762; G16H 30/40; H04N 1/40012; G06T 2207/30024

(57) ABSTRACT

This invention discloses a method and system for the segmentation and clustering of nuclei based on single-cell pathological images. The contour tracing method is used to calculate all the closed contours existing in the pathological tissue images; and optimizes the contours to obtain the segmented images of the nuclei. Then, according to the mask image corresponding to the segmented images of the nuclei, the segmented images of the nuclei are divided into individual nucleus images; the influencing features of the nuclei in the corresponding area are extracted through the mask image; through feature selection, redundant features are removed, and then the UMAP feature reduction method is used to select the two most important features for clustering the nuclei. By first segmenting and dividing the nucleus area in the pathological images, and then using the divided single nucleus for feature extraction, the basis for clustering is made more objective.

### TECHNICAL FIELD

This invention pertains to the field of image processing technology, more specifically, it relates to a method and system for segmentation and clustering of nuclei based on single-cell pathological images.

### BACKGROUND

Segmentation and classification of images are fundamental operations in image processing. Traditionally, these operations are performed using a series of image segmentation functions in Matlab, achieving the goal of segmentation. Subsequently, feature extraction is conducted on the target objects, utilizing the specificity of these features for classification. Although continuous advancements in various research areas have led to significant progress in image segmentation and classification, there remains substantial room for improvement in the precision of pathological image segmentation in the medical field. Moreover, as technology evolves, Computer-Aided Diagnosis (CAD) technologies are increasingly applied across various fields, achieving substantial progress and effects. Thus, the integration of computer technology with medical diagnostics is imminent.

Computer-Aided Diagnosis can provide fast, repeatable medical image analyses, yielding more objective and accurate results. Nucleus detection and segmentation are crucial initial steps in medical image analysis. However, accurate nucleus segmentation and counting still face significant challenges due to uneven staining, irregular cell morphology, adhesion between nuclei, and impurity noise. Current cell image segmentation techniques mainly utilize deep learning algorithms, which require large amounts of training data. Immunohistochemical cell image annotation samples are extremely difficult to obtain, resulting in insufficient training data and, consequently, inadequate accuracy. Additionally, deep learning algorithms are computationally intensive and demand high computer resources.

Therefore, providing a method and system for the segmentation and clustering of pathological image nuclei that can quickly segment cell nuclei and improve the accuracy of nucleus classification is a problem that needs urgent solving by professionals in the field.

### SUMMARY

In light of the above, this invention provides a method and system for the segmentation and clustering of nuclei in single-cell pathological tissue images. By segmenting pathological tissue images into single cells and clustering nuclei based on the segmented individual cells, the accuracy of nucleus classification is improved.

To achieve the aforementioned objectives, the invention offers the following technical solution:

A method for the segmentation and clustering of nuclei in single-cell pathological tissue images, comprising:
reading pathological tissue images;
calculating all closed contours in the pathological tissue images based on a contour tracing method;
evaluating overlapping closed contours based on gradient features of the input pathological tissue images, including the following steps:
obtaining the most prominent local contours; optimizing the closed areas of the most prominent local contours, and segmenting the pathological tissue images based on the optimized contours to obtain nucleus segmented images; dividing the nucleus segmented images into individual nucleus images according to the corresponding mask image;
extracting influencing features of the nuclei in the corresponding area through the mask image;
removing redundant features from the influencing features through feature selection;
and using the UMAP feature reduction method to select the two most important features from the influencing features after feature selection for clustering the nuclei.

Further, the calculation of all closed contours in the pathological tissue images using the contour tracing method specifically involves:

Converting the original color image of the pathological tissue into a grayscale image, by calculating the average values of the RGB channels, thus transforming the pathological tissue image into an image function;

Scanning the grayscale image row by row and storing all local minima and maxima as well as the corresponding maximum gradient between them. Based on these local minima, maxima, and the maximum gradient, determining the starting pixels for contour detection and the corresponding intensity range.

Use an 8-connected neighborhood to follow the contour lines of the object clockwise;

Based on the existing contour pixels, test the adjacent neighborhood clockwise from the starting pixel. If there is a pixel of the current object in the neighborhood, then test the neighborhood in a counterclockwise direction; if the contour also belongs to the current object, then the contour will continue to use that pixel;

Stop when the contour tracing returns to the seed position, and the contour pixel being traced is the same as the second contour pixel;

When the contour returns to its starting pixel, it is considered a valid pixel; if it exceeds the maximum contour length, then the contour tracing is terminated.

Further, evaluate overlapping closed contours based on the gradient features of the input pathological tissue image to obtain the most prominent local contours, specifically by:

Determining the value of each contour based on the following three criteria: (1) Identifying the object with the most prominence and highest average gradient in the same local area; (2) Comparing the gradient fit between contour pixels and the maximum local gradient; (3) Using the Sobel operator and its 3×3 convolution kernel for calculation;

Obtaining the most prominent local contours: Labeling graded isocontours, with the labeling process executed in sorted order, starting from the most valuable contour, and preventing the overwriting of already assigned labels to capture the most prominent local contours.

Further, optimize the closed areas of the most prominent contours to obtain nucleus segmented images, specifically by:
Testing the compactness of object pixels based on the distance value d;
Setting up a loop to process pixels with a specific distance value dt, from dt=d−1 to 1; scanning the entire distance map in each cycle; if a pixel pi with a distance value of di=dt does not have a neighbor with a distance value of dt+1, then the distance value di=dt of pixel pi is decreased by 1;
Separating the object at concave boundary points by removing object pixels around the cut line between two concavities, thereby obtaining nucleus segmented images.

Further, divide the nucleus segmented image into individual nucleus images according to the corresponding mask image, specifically by:
Identifying and labeling the connected domains within the mask image;
Drawing the minimum bounding rectangle around the connected domains and dividing them according to the coordinates of the rectangle.

Further, extract the influencing features of the nuclei in the corresponding area through the mask image, specifically by:
Converting the file format of the divided individual nucleus images into nii format;
Determining the image type and feature type to be used for feature extraction;
Extracting the influencing features of the nuclei according to the determined image type and feature type.

Further, remove redundant features through feature selection, specifically using the Wilcoxon rank-sum test.

Further, use the UMAP feature dimensionality reduction method to select the two most important features for clustering the nuclei, specifically as follows:
(1) Arbitrarily select a sample as the first cluster center Z1;
(2) Choose the sample that is the farthest from Z1 as the second cluster center Z2;
(3) Calculate the distance between each sample and all determined cluster centers one by one, and select the smallest distance for each;
(4) Among all the smallest distances, select the largest distance. If this maximum value reaches or exceeds a predetermined fraction of the distance |Z1-Z2|, then the sample that generated the largest distance is defined as a new cluster center, and return to the previous step; if not, the calculation of cluster centers is concluded;
(5) Repeat steps (3) and (4) until no new cluster centers emerge;
(6) Assign samples to the categories represented by the nearest cluster centers, completing the clustering of the nuclei.

A system for segmentation and clustering of nuclei in single-cell pathological images is designed to implement any of the mentioned methods for segmenting and clustering nuclei based on single-cell pathological images. The system includes a nucleus segmentation subsystem and a nucleus clustering subsystem:

The nucleus Segmentation Subsystem includes:
an image reading module for reading pathological tissue images.
a contour extraction module for calculating all closed contours present in the pathological tissue images.
a contour evaluation module for optimizing the closed contours obtained by the contour extraction module and segmenting the pathological tissue images based on the optimized contours to obtain nucleus segmented images.

The nucleus clustering subsystem includes:
a nucleus segmentation module for dividing the nucleus segmented images into individual nucleus images according to the corresponding mask image obtained from the contour evaluation module;
an influencing feature extraction module for extracting influencing features of the nuclei in the corresponding areas using the mask image;
a feature selection module for removing redundant features from the influencing features; and
a nucleus clustering module for using the UMAP feature dimensionality reduction method to select the two most important features from the influencing features after feature selection to cluster the nuclei.

From the technical solutions mentioned above, it is clear that, compared with existing technologies, this invention provides a method and system for segmentation and clustering of nuclei based on single-cell pathological images. Firstly, the method of segmenting and extracting nucleus images provided by this invention can assist in accurately locating and extracting nuclei more rapidly than machine learning and neural network approaches, and it does not require extensive training data for learning and training to accurately and swiftly segment and extract individual nuclei. Secondly, the invention constructs algorithms based on the concept of segmenting first and then clustering and quantifies features to reduce redundant features through feature selection. By first segmenting and dividing the nuclear areas in the pathological images, and then performing feature extraction on the segmented single nuclei, the basis for clustering is made more objective.

### BRIEF DESCRIPTION OF THE DRAWINGS

To more clearly illustrate the embodiments of the invention or the technical solutions in the existing technology, the drawings used in the description of the embodiments or existing technology will be briefly introduced below. It is evident that the drawings described below are only embodiments of the invention, and for those skilled in the art, other drawings can be obtained from the provided drawings without creative efforts.

FIG. 1 is a flowchart illustrating the method of segmentation and clustering of nuclei provided by the invention.

FIG. 2 is a schematic diagram of the minimum model method matrix provided in an embodiment of the invention.

FIG. 3 is a schematic diagram of the minimum model method matrix provided in an embodiment of the invention.

FIG. 4A is a schematic diagrams of pathological image lesion area sampling provided in an embodiment of the invention, where a represents Lsil, b represents hsil, and c represents cancer.

FIG. 4B is a schematic diagrams of pathological image lesion area sampling provided in an embodiment of the invention, where a represents Lsil, b represents hsil, and c represents cancer.

FIG. 4C is a schematic diagrams of pathological image lesion area sampling provided in an embodiment of the invention, where a represents Lsil, b represents hsil, and c represents cancer.

FIG. 5A is a schematic diagrams of the sampling image segmentation mask provided in an embodiment of the invention, where a represents Lsil, b represents hsil, and c represents cancer.

FIG. 5B is a schematic diagrams of the sampling image segmentation mask provided in an embodiment of the invention, where a represents Lsil, b represents hsil, and c represents cancer.

FIG. 5C is a schematic diagrams of the sampling image segmentation mask provided in an embodiment of the invention, where a represents Lsil, b represents hsil, and c represents cancer.

FIG. 6A is a schematic diagrams of single nucleus clustering provided in an embodiment of the invention, where a represents Lsil, b represents hsil, and c represents cancer.

FIG. 6B is a schematic diagrams of single nucleus clustering provided in an embodiment of the invention, where a represents Lsil, b represents hsil, and c represents cancer.

FIG. 6C is a schematic diagrams of single nucleus clustering provided in an embodiment of the invention, where a represents Lsil, b represents hsil, and c represents cancer.

FIG. 7 is a framework schematic diagram of the nucleus segmentation and clustering system of the invention.

### DETAILED DESCRIPTION OF PREFERRED EMBODIMENTS

The technical solutions in the embodiments of the present invention will be clearly and completely described below with reference to the accompanying drawings in the embodiments of the present invention. Obviously, the described embodiments are only some of the embodiments of the present invention, rather than all the embodiments. Based on the embodiments of the present invention, all other embodiments obtained by those of ordinary skill in the art without creative efforts fall within the scope of protection of the present invention.

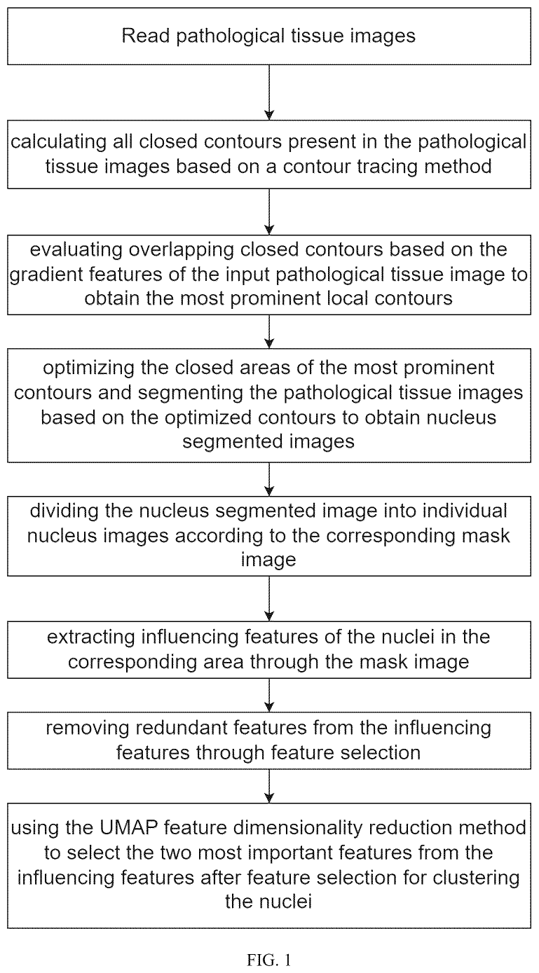

The embodiment of the present invention discloses a method for segmenting and clustering nuclei based on single-cell pathological tissue images, as shown in FIG. 1, including the following steps:

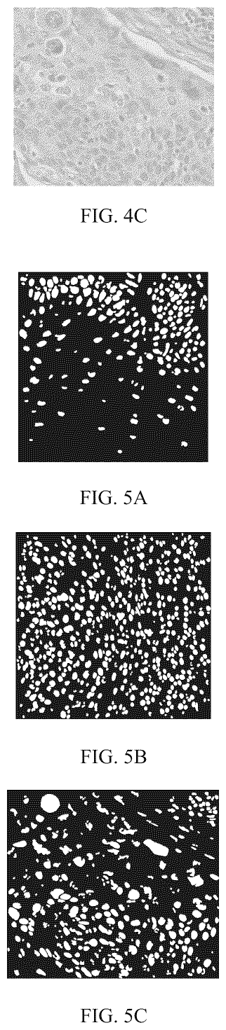

Read pathological tissue images; in this example, 100 pathological images of cervical tumor patients were collected at Xijing Hospital, and 60 pathological images of cervical tumor patients were included after data screening and processing (including 20 pathological images of cervical cancer patients and 20 high-level lesions Pathological images of patients and 20 pathological images of patients with low-grade lesions). Gold standard annotation by professional and experienced gynecologists. The lesion area of the pathological image is sampled, as shown in FIG. 4.

Calculating all closed contours in the pathological tissue images using a contour tracing method.

Evaluate overlapping closed contours based on the gradient features of the input pathological tissue image to obtain the most prominent local contours;

Optimize the closed areas of the most prominent local contours to obtain nucleus segmented images;

Divide the nucleus segmented image into individual nucleus images according to the corresponding mask image;

Extract influencing features of the nuclei in the corresponding area through the mask image;

Remove redundant features through feature selection;

Use the UMAP feature dimensionality reduction method to select the two most important features for clustering the nuclei.

Further, calculate all closed contours in the pathological tissue images using the contour tracing method, specifically by converting the sampled image input into a grayscale image, transforming the image into an image function I(x), scanning the image from left to right row by row, and storing all local minima and maxima as well as the corresponding maximum gradient between them to determine the contour starting pixel detection and corresponding intensity range. Use an 8-connected neighborhood to follow the contour lines of the (potential) object clockwise; as shown in FIG. 2, based on the existing contour pixels, test the adjacent neighborhood clockwise. If there is a pixel belonging to the current object in the neighborhood, then the neighborhood in a counterclockwise direction will also be tested; if the contour belongs to the current object, then the contour will continue using that pixel. Stop when the contour tracing returns to the seed position, and the contour pixel being traced is the same as the second contour pixel; when the contour returns to its starting pixel, it is considered a valid pixel; if it exceeds the maximum contour length (in our example, 225 pixels), then the contour tracing terminates.

Further, evaluate overlapping closed contours based on the gradient features of the input pathological tissue image to obtain the most prominent local contours, specifically by follows.

Determine the most suitable contour according to the following three indicators: (1) Determine the most prominent object with the highest average gradient in the same local area; (2) Compare the gradient fitting between the contour pixels and the maximum local gradient; (3) Use Sobel operator and its 3*3 convolution kernel for calculation. Specifically, to determine if an object is more prominent than other objects in the same local area, a higher average gradient MeanGrandient (1) is more important; to compare the gradient fitting GradientFit (2) between contour pixels and the maximum local gradient, using the Sobel operator S(3) and its 3*3 convolution kernel G are calculated; determine the most suitable contour;

$$MeanGrandient = \frac{{\sum }_{j} \vert S ( {p}_{ij} ) \vert}{{c}_{i}} , \qquad ( 1 )$$

$${p}_{ij}^{max} = \{ \begin{matrix}1 if max \{ \vert S ( {p}_{nm} ) \vert \} ∀ n , m & {x}_{i} - 1 \leq n \leq {x}_{i} + 1 \\ & {y}_{i} - 1 \leq n \leq {y}_{i} + 1 \\ 0 , & otherwise\end{matrix} \}$$

$${GradientFit}_{i} = \frac{{\sum }_{j} {p}_{ij}^{max}}{\vert {C}_{i} \vert} \qquad ( 2 )$$

$$\vert S \vert = \sqrt{{( I * {G}_{x} )}^{2} + {( I * {G}_{y} )}^{2}} \qquad ( 3 )$$

$${G}_{x} = [ \begin{matrix}- 1 & 0 & 1 \\ - 2 & 0 & 2 \\ - 1 & 0 & 1\end{matrix} ]$$

$${G}_{y} = [ \begin{matrix}- 1 & 2 & - 1 \\ 0 & 0 & 0 \\ 1 & 2 & 1\end{matrix} ]$$

Among them, n and m are the number of contours detected horizontally and vertically respectively; Ci is the i-th contour, pij is the j-th contour pixel of the i-th contour; I is the image; Gx and Gy represent the image with horizontal and vertical edge detection respectively.

Obtain the most prominent local contours: mark graded contours. The labeling process is performed in sorted order, starting from the most valuable contours and preventing overwriting of assigned labels. Obtain the most prominent contours locally.

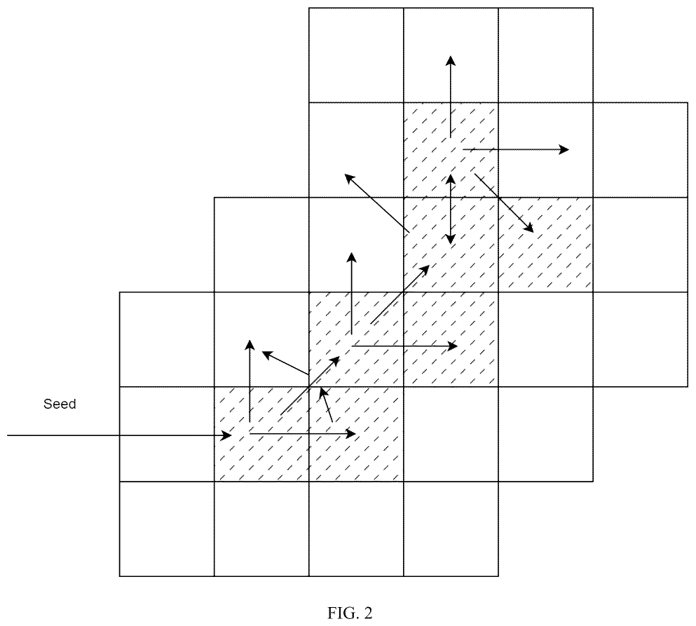

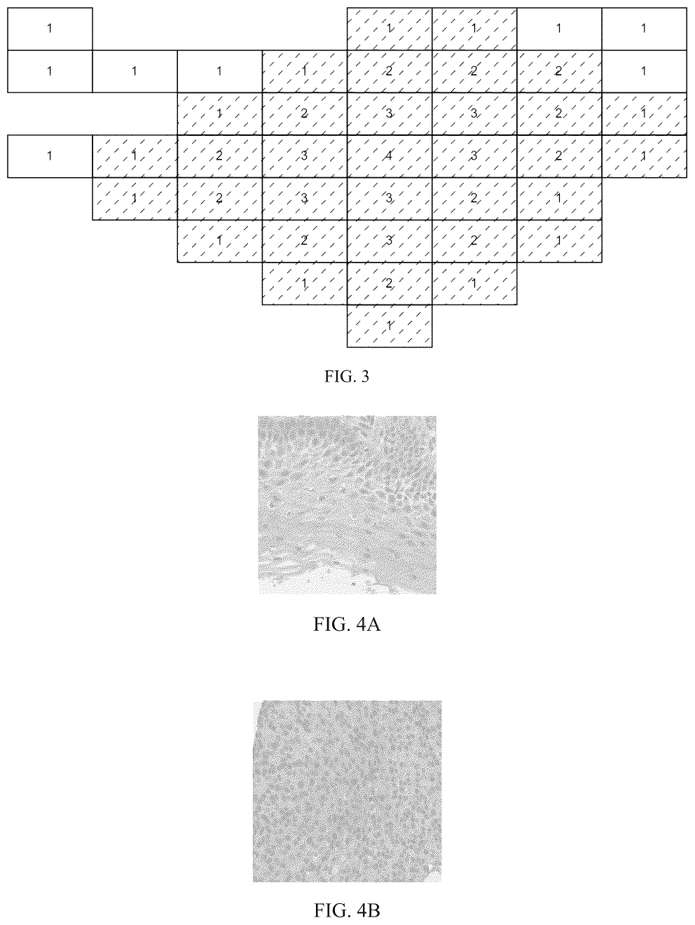

Further, contour optimization is performed on the closed area of the most prominent local contour to obtain the cell nucleus segmentation image, specifically: FIG. 3 shows the compactness of the pixels of the test object based on the distance value d; a loop is set up to process pixels with a specific distance value dt, from dt=d−1 to 1; the entire distance map is scanned in each cycle. If the distance value between pixel pi and di=dt does not have a paraxial neighbor with a distance value of dt+1, the distance value between pixel pi and di=dt will be reduced by 1. Separate objects at the concave boundary by removing object pixels (labels) around the cut line between the two concavities.

Further, the cell nucleus segmentation image is divided into individual cell nucleus small images according to the mask image corresponding to the cell nucleus segmentation image, specifically as follows:

In this embodiment, 100 pathological images of cervical tumor patients were collected, and after data screening and processing, 60 pathological images of cervical tumor patients were included (including 20 pathological images of cervical cancer patients, 20 pathological images of patients with high-level lesions, and 20 pathological images of patients with low-level lesions). Pathological images of diseased patients), and the corresponding 60 mask images were obtained, as shown in FIG. 5.

Identify and label connected domains in the mask image;

Draw the minimum external matrix of the connected domain and segment it by matrix coordinates.

Further, the influencing features of the cell nuclei in the corresponding area are extracted through the mask image, specifically: convert the file format of the segmented small image of a single cell nucleus into nii format; determine the image type and feature type used to extract features; according to the image type and feature types to extract the influencing features of cell nuclei.

Convert the segmented single cell nucleus and the corresponding mask image into a nii format file for feature extraction. The bincount is 100, the label is 1, and the normalizeScale is 255. Use the original image, gradient image, and wavelet image. Image features are divided into the following categories:
1. Shape features describe the shape of the tracking region of interest and its geometric properties, such as volume, maximum diameter along different orthogonal directions, maximum surface, tumor compactness, and sphericity. For example, a needle-shaped tumor will show a higher surface-to-volume ratio than a round tumor of similar volume.
2. First-order statistical features describe the distribution of individual voxel values without considering spatial relationships. These are histogram-based properties that report the mean, median, maximum, and minimum values of voxel intensities on the image, as well as their skewness (asymmetry), kurtosis (flatness), uniformity, and randomness (entropy).
3. Second-order statistical features include so-called texture features, which are obtained by calculating the statistical interrelationships between adjacent voxels. They provide a measure of the spatial arrangement of voxel intensities and are therefore a measure of intralesional heterogeneity. Such features can be derived from the gray level co-occurrence matrix (GLCM), which quantifies the incidence of voxels with the same intensity at a predetermined distance in a fixed direction, or from the gray level run length matrix (GLRLM), which quantifies Continuous voxels with the same intensity in a fixed direction.
4. After filtering or mathematically transforming the image, high-order statistical features are obtained through statistical methods; for example, the purpose is to identify repeated or non-repeating patterns, suppress noise, or highlight details. These methods include fractal analysis, Minkowski functionals, wavelet transforms, and the Laplace transform of Gaussian-filtered images, which can extract regions with increasingly coarse textures.

In order to quantify the spatial heterogeneity within the tumor, the open source radiomics toolbox Pyradiomics was used to extract radiomics features from the ROI of each imaging sequence. The extracted image features are divided into four categories: shape features, first-order statistical features, texture features and wavelet features. Shape features (n=14) represented the ROI shape with a triangular grid to quantitatively evaluate the lesion shape. Its features include flatness, elongation, volume, surface area, maximum diameter, surface-to-volume ratio, density, eccentricity, sphericity, minimum Axis length, second largest axis length. Using first-order statistical features (n=18), through statistical indicators: mean, median, interquartile range, gray value range, minimum value, maximum value, variance, root mean square, skewness, kurtosis, uniformity, and energy, quantitatively describing the distribution of pixel gray levels in ROI. Texture features (n=75) are used to describe the heterogeneity of grayscale distribution in ROI and are found by constructing various grayscale matrices; including 24 grayscale co-occurrence matrices (GLCM), 16 grayscale run length matrices (GLRLM)), 14 grayscale dependency matrices (GLDM), 16 grayscale large zone matrices (GLSZM) and 5 neighborhood grayscale tone difference matrices (NGTDM). To extract more image features that quantify tumor heterogeneity, wavelet filtering and Gaussian filtering were applied to each MRI volume. Wavelet filtering decomposes the original image into 8 decomposition points. Assuming that L and H are low-pass filtering and high-pass filtering respectively, the wavelet decomposition of the image can be marked as LLL, LLH, LHL, LHH, HLL, HLH, HHL, HHH. For example, HHL is obtained by x-direction high-pass filtering, y-direction high-pass filtering, and z-direction low-pass filtering of the original image. The resulting decomposed image has the same size as the original image. For the 8 decomposition methods, 18 first-order statistical features and 75 texture features were calculated respectively, and the corresponding 1023 wavelet features were obtained. Wavelet filtering, gradient filtering, etc. are implemented using the pywavelet package (v. 1.0.1).

Furthermore, redundant features are removed through feature screening, specifically using the Wilcoxon rank sum test. After extracting features, quantify the features. X is the population. Arrange the sample observations with capacity n in order from large to small into x₁, . . . xₙ; each pair was randomly and independently selected; data were examined on at least one interval scale when calculating within-pair differences. The number of samples is N, which is the number of pairs. Therefore, there are a total of 2N data, i=1, . . . , N, x_{1,i} and x_{2,i} represent the measured quantities. H₀: The difference between two pairs follows a symmetric distribution around zero; H₁: The difference between two pairs does not follow a symmetric distribution around zero. The inspection steps are as follows:
1. Calculate |x_{1,i}−x_{2,i}| and sgn(x_{1,i}−x_{2,i}), where sgn is the sign function, and the sign function formula is

$$sgn ( x ) := \{ \begin{matrix}- 1 & if x < 0 , \\ 0 & if x = 0 , \\ 1 & if x > 0.\end{matrix}$$

2. Exclude data with |x_{1,i}−x_{2,i}=0 to reduce the sample size of Nᵣ
3. Sort the remaining data pairs of Nᵣ from the smallest absolute difference to the largest absolute difference
4. The number of the data pair with the smallest absolute difference is 1, and the number increases according to the sorting. Use Rᵢ to represent the rank of the data pair
5. Calculate the verification statistic W, which is the sum of the sequence ranks

$$W = {\sum }_{i = 1}^{{N}_{r}} [ sgn ( {x}_{2 , i} - {x}_{1 , i} ) \cdot {R}_{i} ] \qquad ( 4 )$$

6. Under the assumption H_0, W follows a specific distribution, and the variance is

$$\frac{{N}_{r} ( {N}_{r} + 1 ) ( 2 {N}_{r} + 1 )}{6} .$$

7. If W converges to a normal distribution, for Nᵣ≥20, the z-score is calculated by the

$$formula z = \frac{W}{{σ}_{W}} .$$

$${σ}_{w} = \sqrt{\frac{{N}_{r} ( {N}_{r} + 1 ) ( 2 {N}_{r} + 1 )}{6}} \qquad ( 5 )$$

If Z_{critical}>|z|, it is assumed that H₀ does not hold, or a one-sided test is performed using an exact or approximate distribution, and the p value is calculated to make a statistical conclusion.

For Nᵣ<20 the exact distribution needs to be used.

The WLCX feature selection method uses the Wilcoxon rank sum test method to calculate the p-value between each feature and the label for feature selection, and selects the feature with the highest correlation with the label.

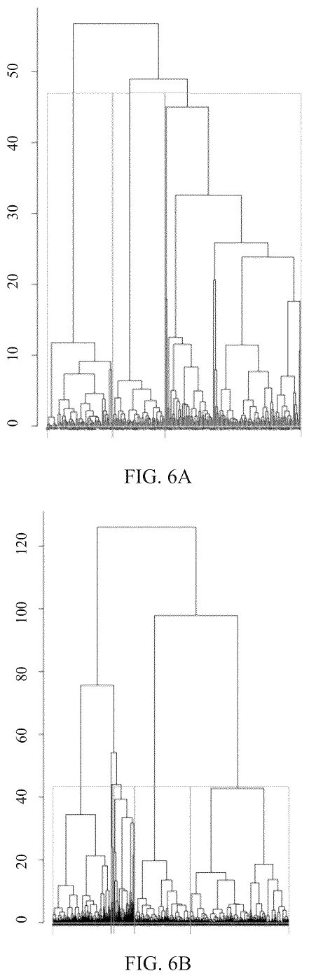

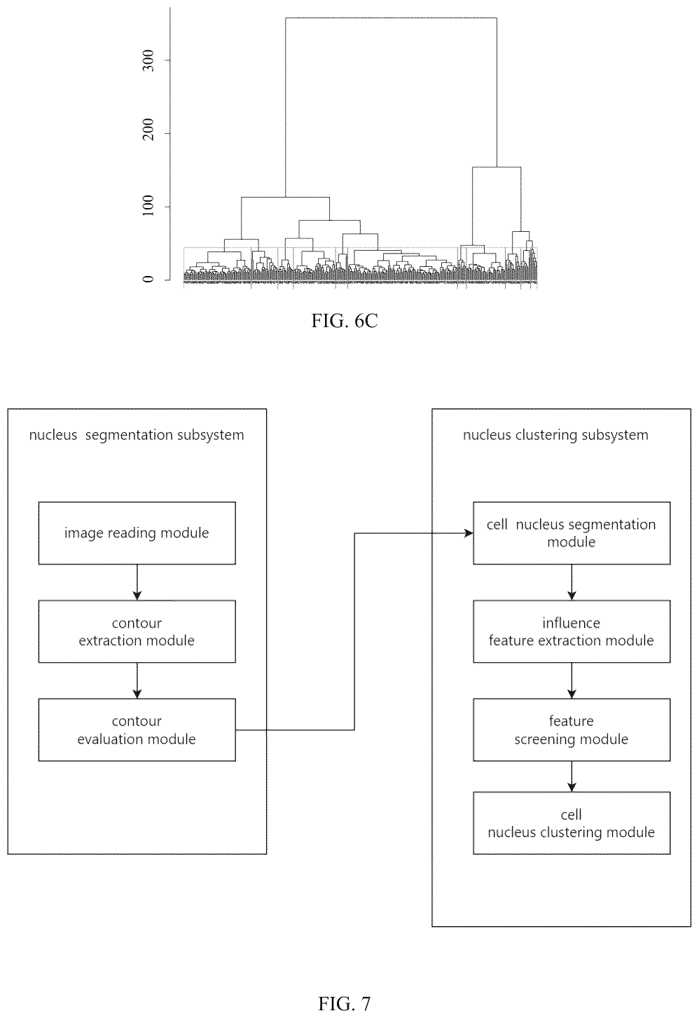

Furthermore, the UMAP feature dimensionality reduction method is used to screen the two most important features to cluster the cell nuclei, specifically:
(1) Select any sample as the first cluster center Z1;
(2) Select the sample farthest from Z1 as the second cluster center Z2;
(3) Calculate the distance between each sample and all known cluster centers one by one, and select the minimum distance among them;
(4) Select a maximum distance among all minimum distances. If the maximum value reaches the preset score ratio of ∥Z1−Z2∥, the sample that produces the maximum distance is defined as the new cluster center and returns to the previous step. Otherwise, the calculation step of cluster center ends;
(5) Repeat steps (3) and (4) until no new clustering center appears;
(6) Divide the samples into the categories represented by the corresponding cluster centers according to the closest distance to complete the clustering of cell nuclei. As shown in FIG. 6, the Lsil sampling map is clustered into 3 categories, the hsil sampling map is clustered into 5 categories, and the cancer sampling map is clustered into 11 categories.

The present invention also provides a segmentation and clustering system based on the nuclei of single-cell pathological images, which is used to implement a segmentation and clustering method based on the nuclei of single-cell pathological images. As shown in FIG. 7, it specifically includes: a cell nucleus segmentation sub-system and a nuclear clustering subsystems.

The cell nucleus segmentation subsystem includes:
an image reading module used to read pathological tissue images;
a contour extraction module used to calculate closed contours present in pathological
tissue images; and
a contour evaluation module used to optimize the closed contour obtained by the contour extraction module, and segment the pathological tissue image based on the optimized contour to obtain the cell nucleus segmentation image;

The cell nucleus clustering subsystem includes:
a cell nucleus segmentation module used to segment the cell nucleus segmentation image into single small cell nucleus images based on the cell nucleus segmentation image and the corresponding mask image obtained by the contour evaluation module;
an influence feature extraction module used to extract the influence features of the cell nuclei in the corresponding area based on the mask image;
a feature screening module, used to remove redundant features from influencing features;
a cell nucleus clustering module used to use the UMAP feature dimensionality reduction method to select the two most important features among the influencing features that have been screened to cluster the cell nuclei.

The cell nucleus clustering subsystem also includes a display module for displaying the clustering results of the cell nucleus clustering module.

Each embodiment in this specification is described in a progressive manner. Each embodiment focuses on its differences from other embodiments. The same and similar parts between the various embodiments can be referred to each other. As for the device disclosed in the embodiment, since it corresponds to the method disclosed in the embodiment, the description is relatively simple. For relevant details, please refer to the description in the method section.

The above description of the disclosed embodiments enables those skilled in the art to make or use the present invention. Various modifications to these embodiments will be readily apparent to those skilled in the art, and the generic principles defined herein may be practiced in other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for the segmentation and clustering of nuclei based on single-cell pathological images, characterized in that, comprising:
reading pathological tissue images;
calculating all closed contours present in the pathological tissue images based on a contour tracing method;
evaluating overlapping closed contours based on the gradient features of the input pathological tissue image to obtain the most prominent local contours;
optimizing the closed areas of the most prominent contours and segmenting the pathological tissue images based on the optimized contours to obtain nucleus segmented images;
dividing the nucleus segmented image into individual nucleus images according to the corresponding mask image;
extracting influencing features of the nuclei in the corresponding area through the mask image;
removing redundant features from the influencing features through feature selection; and
using the UMAP feature dimensionality reduction method to select the two most important features from the influencing features after feature selection for clustering the nuclei.

2. The method of claim 1, characterized in that, the calculating all closed contours present in the pathological tissue images based on a contour tracing method comprises:
converting the original color image of the pathological tissue image into a grayscale image, calculating the average of the three RGB channels of the image, and converting the pathological tissue image into an image function;
scanning the grayscale image line by line and storing all local minimum values and local maximum values and the corresponding maximum gradient between them, and determining a starting pixel and corresponding intensity range of the contour detection based on the local minimum value, the maximum value and the maximum gradient;
using an 8-connected neighborhoods to follow the contours of the tracking object clockwise;
testing a paraxial neighborhood clockwise from the starting pixel on the basis of the existing contour pixels, if there is a pixel of the current object in the neighborhood, testing an counterclockwise neighborhood; if the contour also belongs to the current object, then the contour continuing to using the pixel;
stopping tracking when the contour tracking returns to a seed position, and the contour pixel that continues to be tracked is the same as a second contour pixel;
when the contour returns to its starting pixel, marking the starting pixel as a valid pixel; if the maximum contour length is exceeded, contour tracing being terminated.

3. The method of claim 1, characterized in that, the evaluating overlapping closed contours based on the gradient features of the input pathological tissue image to obtain the most prominent local contours comprises:
determining the value of each contour based on the following three criteria: (1) identifying the object within the same local area that is the most prominent and has the highest average gradient; (2) comparing the gradient fit between contour pixels and the maximum local gradient variation; (3) utilizing the Sobel operator and its 3×3 convolution kernel for calculation; and
obtaining the most prominent local contours involves labeling graded contour lines, with the labeling process conducted in a sorted order, starting from the most valuable contour and preventing the overwriting of already assigned labels to obtain the most prominent local contours.

4. The method of claim 1, characterized in that the step of performing contour optimization comprises:
testing the compactness of object pixels based on the distance value d;
establishing a loop to process pixels with a specific distance value dt, from dt=d−1 to 1; each cycle requiring scanning the entire distance map, if a pixel pi with a distance value of di=dt does not have a neighbor with a distance value of dt+1, then the distance value di=dt of pixel pi is decreased by 1;
separating objects at concave boundary points by removing object pixels around the cut line between two concavities to obtain the nucleus segmented image.

5. The method of claim 1, characterized in that the step of dividing the cell nucleus segmentation image into individual cell nucleus small images comprises:
identifying and labeling the connected domains within the mask image; and
drawing the minimum bounding rectangles of the connected domains and dividing them through the coordinates of these rectangles.

6. The method of claim 1, characterized in that the step of extracting the influence features of the cell nucleus comprises:
converting the file format of the segmented small image of a single cell nucleus to nii format;
determining the image type and feature type used to extract features;
extracting the influencing features of the cell nucleus according to the image type and feature type.

7. The method of claim 1, characterized in that Wilcoxon rank sum test is used to remove redundant features from influencing features through feature screening.

8. The method of claim 1, characterized in that step of using the UMAP feature dimensionality reduction method to select the two most important features among the influencing features comprises:
(1) selecting any one of the cell nuclei as the first clustering center Z1;
(2) selecting the cell nucleus farthest from Z1 as the second clustering center Z2;
(3) calculating the distance between each sample and all known cluster centers one by one, and select the minimum distance among them;
(4) selecting a maximum distance among all minimum distances, if the maximum value reaches more than the preset score ratio of |Z1−Z2|, then the cell nucleus that produces the maximum distance is defined as a new clustering center and returns previous step; otherwise, the calculation step of cluster center ends;
(5) repeating steps (3) and (4) until no new clustering center appears;
(6) dividing the cell nuclei into the categories represented by the corresponding cluster centers according to the nearest distance to complete the clustering of cell nuclei.

9. A segmentation and clustering system based on the nuclei of single-cell pathological images, used to implement the method of claim 1, characterized in that, the system comprises a nucleus segmentation subsystem and a nucleus clustering subsystem;
the cell nucleus segmentation subsystem comprising:
an image reading module, used to read pathological tissue images;
a contour extraction module, used to calculate the closed contour existing in the pathological tissue image; and
a contour evaluation module, configured to optimize the closed contour obtained by the contour extraction module, and segment the pathological tissue image based on the optimized contour to obtain a cell nucleus segmentation image;
the cell nucleus clustering subsystem comprising:
a cell nucleus segmentation module, configured to segment the cell nucleus segmentation image into individual cell nucleus small images based on the cell nucleus segmentation image and the corresponding mask image obtained by the contour evaluation module;
an influence feature extraction module, used to extract the influence feature of the cell nucleus in the corresponding area according to the mask image;
a feature screening module, used to remove redundant features from influencing features;
a cell nucleus clustering module, used to use the UMAP feature dimensionality reduction method to select the two most important features among the influencing features that have been screened to cluster the cell nuclei.
